(19) United States
(12) Patent Application Publication
(10) Pub. No.: US 2013/0251790 A1
(43) Pub. Date: Sep. 26, 2013

(54) Low Dose Pharmaceutical Composition Comprising Zanamivir

(71) Applicants: Geena Malhotra, Mumbai (IN); Shrinivas Madhukar Purandare, Mumbai (IN)

(72) Inventors: Geena Malhotra, Mumbai (IN); Shrinivas Madhukar Purandare, Mumbai (IN)

(73) Assignee: CIPLA LIMITED, Mumbai (IN)

(21) Appl. No.: 13/825,469

(22) PCT Filed: Sep. 26, 2011
(86) PCT No.: PCT/GB11/01395

§ 371 Date: May 28, 2013

(30) Foreign Application Priority Data
Sep. 27, 2010 (IN) 2684/MUM/2010

(51) Int. Cl. A61K 31/351; A61M 15/00; A61K 45/06

(52) U.S. Cl. CPC A61K 31/351; A61K 45/06; A61M 15/0028
USPC 424/451; 514/459; 514/263.31; 514/50; 514/46; 514/43; 514/40; 514/200; 514/209; 514/3.7; 128/203.21

(57) ABSTRACT

The present invention provides a pharmaceutical composition comprising a low dose of zanamivir and a process for preparing the pharmaceutical composition comprising a low dose of zanamivir. The pharmaceutical composition comprising a low dose zanamivir may be used in the treatment and/or prophylaxis of influenza. The present invention also provides a method of treatment and/or prophylaxis of influenza which comprises administering a dry powder inhaler composition comprising a low dose zanamivir. The pharmaceutical composition of the present invention comprises zanamivir and one or more pharmaceutically acceptable excipients, wherein the total daily dose of the zanamivir is less than 10 mg, preferably for administration at least once a day, and preferably wherein the composition delivers from 3 mg to 8 mg of zanamivir per administered dose.

### CROSS-REFERENCE TO RELATED APPLICATIONS

The present invention claims the benefit of the PCT/GB2011/000428 filed Sep. 30, 2011, which claims priority to Ser. No. 2750/MUM/2010 filed Oct. 1, 2010.

### FIELD OF INVENTION

The present invention relates to a novel low dose pharmaceutical composition comprising an antiviral drug. The present invention also provides a process of preparing such pharmaceutical composition and its use in the treatment and/or prophylaxis of influenza.

### BACKGROUND OF INVENTION

Influenza is a common condition affecting all age groups. It occurs mainly during the winter season and causes significant morbidity and increased mortality. The elderly and those with pre-existing medical problems, such as heart disease and renal disease, are particularly at the risk of suffering this severe disease or developing such complications.

Neuraminidase inhibitors are a class of antiviral drugs specifically targeted at the influenza virus, which act by blocking the function of the viral neuraminidase protein, thereby preventing the virus from reproducing by budding method from the host cell.

Zanamivir is the first such neuraminidase inhibitor to be developed commercially, and is used in the treatment of and prophylaxis of both Influenza virus A as well as Influenza virus B. The chemical name of zanamivir is 5(acetylamino)-4-[(aminoiminomethyl)-amino]-2,6-anhydro-3, 4, 5-trideoxy-D-glycero-D-galactonon-2-enonic acid.

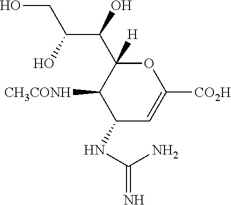

Zanamivir is a selective inhibitor of neuraminidase, an enzyme that cleaves sialic acid from host and viral cell surfaces and thereby facilitates the release of progeny virus from infected host cells. Zanamivir also prevents neuraminidase from cleaving sialic acid from the host cells by blocking the active site of neuraminidase. The resultant binding of viral hemagglutinin to the uncleaved sialic acid hinders release of nascent viruses from the host cell and thereby causes them to clump at the host cell surface, with a net reduction in the amount of active virus.

Zanamivir has been approved for treatment and prophylaxis of illness due to influenza A and B virus in adults and in pediatric patients. Zanamivir is administered by oral inhalation. This mode of administration delivers drug directly to the pulmonary site of the influenza infection and thereby minimizes its systemic exposure.

The recommended therapeutic dose of zanamivir for treatment of influenza in adults as well as pediatric patients is 10 mg twice daily for 5 days. Further, the recommended therapeutic dose of zanamivir for prophylaxis of influenza in adults and pediatric patients in a household setting is 10 mg twice daily for 10 days and the recommended therapeutic dose of zanamivir for prophylaxis of influenza in adults and pediatric patients in a community setting is 10 mg once daily for 28 days.

EP0764023 discloses administration of zanamivir by mouth via inhalation or insufflation.

JP2002241310 discloses locally applicable compositions in the form of nasal drops and inhalants containing influenza virus inhibitors such as zanamivir.

CN101229122 discloses formulations of zanamivir in a nasal in-situ gel with advantages of improved concentration of zanamivir in respiratory tracts, long drug detention time, high bioavailability, improved patient compliance, and no nasal ciliary toxicity.

CN101773468 discloses nasal nanoscale suspensions of zanamivir with advantages of low dosage, long mucous membrane detention time, and good curative effects.

CN101773491 discloses a zanamivir inhalation solution.

Various formulations that are disclosed and that are available in the market contain a dose of 5 mg of zanamivir for twice a day administration. However, no composition is yet available which includes low dose zanamivir, wherein the total daily dose of zanamivir is less than the conventionally administered daily dose of at least about 10 mg of zanamivir, and which is still effective for the prophylaxis and/or treatment of influenza.

Hence, there still exists a need to develop pharmaceutical compositions comprising a low dose of zanamivir, wherein the total daily dose of zanamivir is less than the conventionally administered daily dose of zanamivir, and which also does not require the use of any specific bioenhancer and which can release the drug in a desired manner and in a quantity sufficient to alleviate desired pathological conditions without causing or at least minimizing dose related toxicity, and can be prepared in an easy and cost-effective manner.

### OBJECT OF THE INVENTION

An object of the present invention is to provide a novel low dose pharmaceutical composition comprising zanamivir along with one or more pharmaceutically acceptable excipient(s).

Another object of the present invention is to provide a novel low dose pharmaceutical composition comprising zanamivir wherein the total daily dose of zanamivir is less than the conventionally administered daily dose of at least about 10 mg of zanamivir.

Yet another object of the present invention is to provide a process for preparing the pharmaceutical composition comprising a low dose of zanamivir for administration in the treatment or prophylaxis of influenza.

A further object of the present invention is to provide a method for treatment and/or prophylaxis of influenza which comprises administering a pharmaceutical composition comprising a low dose of zanamivir.

### SUMMARY OF THE INVENTION

According to one aspect of the present invention, there is provided a pharmaceutical composition comprising a low dose of zanamivir.

According to another aspect of the present invention there is provided a process for preparing the pharmaceutical composition comprising a low dose of zanamivir.

According to yet another aspect of the present invention there is provided a pharmaceutical composition comprising a low dose zanamivir for use in the treatment and/or prophylaxis of influenza.

According to a further aspect of the present invention there is provided a method of treatment and/or prophylaxis of influenza which comprises administering a dry powder inhaler composition comprising a low dose zanamivir.

### BRIEF DESCRIPTION OF THE FIGURES

FIG. 1 is a cross-sectional view of the inhalation device, with piercing means in a fully retracted position and the inhaler mouthpiece in a fully opened position.

FIG. 2 is a cross-sectional view of the inhalation device, with piercing means in a fully extended piercing position and the inhaler mouthpiece in a partially open/partially closed position, and illustrates a preferred position of the vents.

FIG. 3 is a cross-sectional view of the inhalation device, with piercing means in a fully retracted position and the inhaler mouthpiece in a fully closed position.

FIG. 4 is a perspective view of the exterior of the inhalation device with the mouthpiece in the fully closed position.

FIG. 5 is a plan view of a side of the inhalation device with the mouthpiece in the fully closed position.

FIG. 6 is a plan view of the other side (with respect to FIG. 5) of the inhalation device with the mouthpiece in the fully closed position.

FIG. 7 is a plan view of the rear of the inhalation device with the mouthpiece in the fully closed position.

FIG. 8 is a plan view of the front of the inhalation device with the mouthpiece in the fully closed position.

FIG. 9 is a top end view of the inhalation device with the mouthpiece in the fully closed position, viewed from the mouthpiece end.

FIG. 10 is a bottom end view of the inhalation device with the mouthpiece in the fully closed position, viewed from the lower body end.

FIG. 11 is a perspective view of a holder for a dry powder medicament capsule.

FIG. 12 is an end view of the medicament capsule holder, viewed from the air outlet end.

FIG. 13 is an end view of the medicament capsule holder viewed from the air inlet end.

FIG. 14 is a front view of the medicament capsule holder.

FIG. 15 is a side view of the medicament capsule holder.

FIG. 16 is a rear view of the medicament capsule holder.

FIG. 17 is a side view of the medicament capsule holder.

FIG. 18 is a top end view of a preferred inhalation device with the mouthpiece in the fully closed position, viewed from the mouthpiece end. It illustrates the position of the vents.

FIG. 19 is a cross-section of a preferred inhalation device illustrating projections

(16) of increased height compared to FIG. 11.

FIG. 20 is a cross-section of a preferred inhalation device and shows a preferred position of the vents.

### DETAILED DESCRIPTION OF THE INVENTION

Zanamivir has been administered conventionally by oral inhalation as 5 mg twice a day for the treatment and/or prophylaxis of illness due to influenza A as well as influenza B.

The inventors of this invention have made an effort to provide a lower dose of zanamivir, which lower dose can also be effectively administered for the treatment and/or prevention of influenza. Furthermore, the low dose compositions of the present invention have improved bioavailability, and are easy to formulate. Preferably, the compositions of the present invention do not require the use of any specific bioenhancer or the like.

The term ‘low dose’ as used herein refers to a therapeutically effective dose of zanamivir, which dose is less than the usual or the conventional dose required to produce the therapeutic effect.

The term “Zanamivir” is used in broad sense to include not only “Zanamivir” per se but also their pharmaceutically acceptable salts, pharmaceutically acceptable solvates, pharmaceutically acceptable hydrates, pharmaceutically acceptable esters, pharmaceutically acceptable enantiomers, pharmaceutically acceptable derivatives, pharmaceutically acceptable polymorphs, pharmaceutically acceptable prodrugs, pharmaceutically acceptable complexes etc.

The present invention provides novel low dose pharmaceutical compositions comprising zanamivir or its pharmaceutically acceptable salts, esters, prodrugs, solvates, hydrates, or derivatives thereof, along with one or more pharmaceutically acceptable excipient(s).

According to one aspect of the present invention, there is provided a pharmaceutical composition comprising zanamivir with one or more pharmaceutically acceptable excipients wherein the total daily dose of the zanamivir is less than 10 mg.

The pharmaceutical composition, according to the present invention may be administered at least once a day. Preferably the pharmaceutical composition is administered once a day or twice a day in a dose which is less than the conventionally administered dose, which is about 5 mg of zanamivir twice a day.

According to an embodiment of the present invention provides a novel low dose pharmaceutical composition comprising zanamivir wherein the dose of the zanamivir is in a range of 3 mg to 8 mg, for administration at least once a day, and wherein the total daily dose of zanamivir is less than the conventionally administered daily dose of at least about 10 mg of zanamivir. Therefore, the pharmaceutical composition of the present invention may provide a dose of <10 mg zanamivir for once a day administration or a dose of <5 mg for twice a day administration. The pharmaceutical composition of the present invention may provide a dose of ≦8 mg, or a dose of ≦7 mg, or a dose of ≦6 mg, or a dose of ≦5 mg, or a dose of ≦4 mg, or a dose of ≦3 mg.

The pharmaceutical composition, according to the present invention, may exhibit bioavailability to an extent to produce the desired pharmacological effects along with reduced side effects after dosing in a subject.

The pharmaceutical composition, according to the present invention, may be used for the treatment and/or prophylaxis of viral diseases caused by orthomyxoviruses and paramyxoviruses such as, but not limited to, influenza A and B, parainfluenza, mumps, and Newcastle disease, fowl plaque and Sendai virus.

The pharmaceutical composition, of the present invention, may be administered by any suitable methods used for delivery of the drugs to the respiratory tract. The composition of the present invention may thus be administered as metered dose inhalers (MDI), dry powder inhalers (DPI), solution or suspension for nebuliser, nasal spray, nasal drops, insufflation powders.

The various dosage forms according to the present invention may comprise carriers/excipients suitable for formulating the same.

According to one embodiment the pharmaceutical composition of the present invention may be administered by a dry powder inhaler (DPI).

The pharmaceutically acceptable excipients suitable for dry powder inhalation according to the present invention, may be selected from suitable carriers which comprise, but are not limited to, sugars such as glucose, saccharose, lactose and fructose, starches or starch derivatives, oligosaccharides such as dextrins, cyclodextrins and their derivatives, polyvinylpyrrolidone, alginic acid, tylose, silicic acid, cellulose, cellulose derivatives (for example cellulose ether), sugar alcohols such as mannitol or sorbitol, calcium carbonate, calcium phosphate, lactose, lactitol, dextrates, dextrose, maltodextrin, saccharides including monosaccharides, disaccharides, polysaccharides; sugar alcohols such as arabinose, ribose, mannose, sucrose, trehalose, maltose and dextran.

The pharmaceutical, composition according to the present invention, may be included in suitable containers provided with means enabling the application of the contained formulation to the respiratory tract.

The powder for inhalation intended to be used for DPI may either be encapsulated in capsules of gelatin or hydroxypropyl methylcellulose or in blisters or alternatively, the dry powder may be contained as a reservoir either in a single dose or multi-dose dry powder inhalation device.

According to one embodiment of the present invention, the pharmaceutical composition of the present invention, intended to be used as a DPI may be administered via Revolizer™.

Preferred embodiments of our Revolizer™ are described in WO 2006/051300 and WO 2007/144659 and online at revolizer.com. In its broadest aspect, the Revolizer™ is an inhalation device for inhalation of a medicament from a pierceable capsule, which inhaler comprises a housing for receiving a medicament capsule; closure means for closing the housing, said closure means being moveable relative to the housing; piercing means suitable for piercing a medicament capsule; wherein movement of the closure means relative to the housing causes movement of the piercing means. The Revolizer™ inhalation device may also comprise linking means connected to both the closure means and the piercing means, wherein movement of the closure means causes movement of the linking means so as to move the piercing means. Preferably, movement of the closure means causes movement or rotation of the linking means.

Thus, in use, piercing or perforation of a capsule received in the housing occurs as a consequence of merely closing the housing subsequent to having placed a medicament-containing capsule therein. The user is not required to perform any additional actions prior to inhalation of medicament from the inhaler device, other than inserting a capsule and closing the device.

We have found that the inclusion of one more vents enables the patient to inhale more easily, because of a reduced resistance to air flow. In other words, the perceived resistance to inhalation experienced by the user is lower.

It will be appreciated that the Revolizer™ inhalation device will comprise at least one main primary air inlet through which air is initially drawn from the atmosphere into the device. Likewise, the device will also comprise a final air outlet through which inhaled air exits the device and enters the patient's mouth. Between this initial air inlet and final air outlet, the device will define a passageway through the body of the device through which air flows. The passageway is referred to herein as the inhalation passage. In preferred embodiments, the inhalation passage of the Revolizer™ inhalation device comprises one or more vents, which it will be understood are essentially auxiliary air inlets. The vents may be positioned at one or more points along the inhalation passage and are essentially small openings in the wall of the inhalation passage, which serve to connect air in the inhalation passage with air external to the passage.

Preferably, two vents, which suitably oppose each other, are employed, although one vent, or more than two vents may be employed if desired. The vents can be of any suitable shape and size, although they are preferably crescent-shaped.

It is preferred to position these vents upstream of the medicament capsule, relative to the flow of air through the inhalation passage.

The housing preferably comprises means to hold a medicament capsule; said holding means preferably comprising a chamber having one or more air inlets and air outlet(s). The air inlet of the chamber may constitute the primary air inlet of the device, or the primary air inlet of the device may be provided separately on the device, for example elsewhere on the housing. The or each air inlet and outlet are preferably provided at opposing ends of the chamber. In a preferred embodiment, the or each air inlet is positioned in, or near to, the base of the medicament holder. They may, for example, be provided on the walls of the chamber, for example on the lower walls. The flow of air via the or each inlet may be at a tangent to, or at an angle offset from, the longitudinal axis of the chamber. In one embodiment, a single air inlet may be used. Alternatively, two or more inlets may be used. For example, two air inlets may be provided at or near the bottom of the holder. Preferably, they are provided in the lower vertical walls of the chamber. For example, two inlets may be provided substantially opposite to one another in the lower walls. These inlets may, if desired, be offset from one another; for example in a tangential arrangement.

The closure means preferably comprises a mouthpiece. That is to say, the closure means is preferably such that it includes means by or via which medicament may be inhaled from the device by the user. An inhaler mouthpiece is preferably pivotably attached to the housing.

The air outlet of the medicament chamber is suitably positioned so as to connect or coincide with the mouthpiece in its fully closed position. For example, the mouthpiece may suitably comprise means to receive air from an outlet of the medicament chamber, such that when the mouthpiece is closed the means for receiving air is connected with, or cooperates with, the outlet. Suitably, the means (e.g. a tube which may be cylindrical or oval shaped in cross section) comprises one end which connects with the outlet of the medicament chamber, and another end which comprises an outlet for the medicament, from which outlet medicament is inhaled by the user. It is preferred that the end which connects with the outlet of the medicament chamber comprises the vents, preferably two opposing crescent-shaped vents positioned at either side. The arrangement is preferably such that the vents are in close proximity to the outlet of the medicament chamber. The outlet of the said means may constitute the final air outlet of the inhaler device. Preferably, the positioning, in particular the angle, of the vents is such that in use the direction of airflow through the vents is in the same general direction as the direction of the main inhalation airflow through the inhalation passage. As described above, the vents are typically formed as a hole or opening through the thickness of one or more walls of the inhalation passage. Where the wall thickness is such that an axis through the vent can be defined, the axis of the vent is preferably at an angle of less than 90 degrees, more preferably 45 degrees or less, relative to the direction of the main inhalation airflow through the inhalation passage. That is to say, the flow of air through the vents into the inhalation passage is preferably not perpendicular to, or against, the main inhalation flow, but flows in the same general direction.

The Revolizer™ inhalation device comprises a holder for a medicament, which holder comprises a chamber suitable for receiving a medicament capsule; and means for generating turbulence in a fluid flow through the chamber such that, in use, the turbulent fluid flow causes vibration of a capsule received by the chamber so as to assist in releasing medicament contained within the capsule. Maximizing the turbulence in the chamber enhances drug dispersion and delivery.

The means for generating turbulence preferably also holds, or partially holds, a medicament capsule within the holder. Optionally, the means for generating turbulence holds one end of a medicament capsule. Preferably the capsule is held loosely (either completely, partially or at one end), which means that any significant movement of the capsule is prevented but the capsule is still enabled to make small vibratory movements within the chamber.

The means for generating turbulence may be any suitable means but preferably comprises one or more projections or flow barriers extending from the inner walls of the chamber. Preferably, the means for generating turbulence extend substantially the entire length of the chamber—that is, substantially from top to bottom. Increasing the height of the means for generating turbulence results in reduced retention of medicament in the chamber, and also reduced leakage of medicament from around the top of the chamber.

According to another embodiment of the present invention, the pharmaceutical composition of the present invention may be administered by a metered dose inhaler (MDI).

The metered dose inhalers, according to the present invention, may comprise one or more pharmaceutically acceptable excipients such as hydrofluoroalkane (HFC/HFA) propellants, co-solvents, bulking agents, non volatile component, buffers/pH adjusting agents, surface active agents, preservatives, complexing agents, or combinations thereof.

Propellants are those which, when mixed with the cosolvent(s), form a homogeneous propellant system in which a therapeutically effective amount of the active can be dissolved. The hydrofluoroalkane HFC/HFA propellant must be toxicologically safe and must have a vapor pressure which is suitable to enable the medicament to be administered via a pressurized MDI.

According to the present invention, the HFC/HFA propellants may comprise, but are not limited to, one or more of 1,1,1,2-tetrafluoroethane (HFC-134(a)) and 1,1,1,2,3,3,3-heptafluoropropane (HFC-227), HFC-32 (difluoromethane), HFC-143(a) (1,1,1-trifluoroethane), HFC-134 (1,1,2,2-tetrafluoroethane), and HFC-152a (1,1-difluoroethane) and such other propellants which may be known to the person a skilled in the art.

Co-solvent is any solvent which may be volatile or non-volatile, which is miscible in the formulation in the amount desired and which, when added provides a formulation in which the medicament can be dissolved or suspended. The function of the cosolvent is to increase the solubility of the medicament and the excipients in the formulation.

According to the present invention, the volatile and non-volatile co-solvent may comprise one or more of, C₂₋C₆aliphatic alcohols, such as but not limited to, ethyl alcohol and isopropyl alcohol; glycols such as but not limited to, propylene glycol, polyethylene glycols, polypropylene glycols, glycol ethers, and block copolymers of oxyethylene and oxypropylene; and other substances, such as but not limited to, polyoxyethylene alcohols, and polyoxyethylene fatty acid esters; hydrocarbons such as but not limited to, n-propane, n-butane, isobutane, n-pentane, iso-pentane, neo-pentane, and n-hexane; and ethers such as but not limited to, diethyl ether, monosaccharides such as but not limited to, glucose, arabinose; disaccharides such as lactose, maltose; oligosaccharides and polysaccharides such as but not limited to, dextrans; polyalcohol such as but not limited to, glycerol, sorbitol, mannitol, xylitol; salts such as but not limited to, potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate and calcium chloride.

Suitable buffers or pH adjusting agents may be employed in the MDI formulation of the present invention.

According to the present invention, the buffer or the pH adjusting agent may comprise one or more of organic or inorganic acids such as but not limited to, citric acid, ascorbic acid, hydrochloric acid, sulfuric acid, nitric acid, or phosphoric acid.

Suitable preservatives may be employed in the MDI formulation of the present invention to protect the formulation from contamination with pathogenic bacteria.

According to the present invention, the preservative may comprise, but not limited to, one or more of benzalkonium chloride, benzoic acid, benzoates such as sodium benzoate and such other preservatives which may be known to the person skilled in the art.

Suitable complexing agents may also be employed in the MDI formulation of the present invention which are capable of forming complex bonds.

According to the present invention, the complexing agent may comprise one or more of, but not limited to sodium EDTA or disodium EDTA.

Suitable surfactants may also be employed in the MDI formulation of the present invention which serve to stabilize the solution formulation and improve the performance of valve systems of the metered dose inhaler.

According to the present invention, the surfactant may comprise one or more ionic and/or non-ionic surfactant, but not limited to, oleic acid, sorbitan trioleate, lecithin, isopropylmyristate, tyloxapol, polysorbates such as polysorbate 80, vitamin E-TPGS, and macrogol hydroxystearates such as macrogol-15-hydroxystearate.

Optionally, the powder for inhalation intended to be used for DPI may be suspended in a suitable vehicle and packaged in a container along with suitable propellants or mixtures thereof to form a composition suitable for administration by MDI.

Suitable vehicles which may be used in the pharmaceutical composition of the invention may comprise, but are not limited to, polar solvents, such as compounds that contain hydroxyl groups or other polar groups. Such solvents may include water or alcohols, such as ethanol, isopropanol, and glycols including propylene glycol, polyethylene glycol, polypropylene glycol, glycol ether, glycerol and polyoxyethylene alcohols.

According to a further embodiment, the pharmaceutical composition of the present invention may be administered by nebulization.

The pharmaceutical composition to be administered by a nebulizer, according to the present invention may comprise suitable excipients such as, but not limited to, tonicity agents, surfactants or wetting agents, buffers/pH regulators, chelating agents in a suitable vehicle such as water.

Suitable surfactants or wetting agents may also be used in the pharmaceutical compositions of the present invention. According to the present invention, the surfactants may comprise one or more of, but not limited to polysorbates such as polysorbate 20, polysorbate 40, polysorbate 60, polysorbate 80, polysorbate 65, polysorbate 85, sorbitan, fatty acid esters such as Span 20, Span 40, Span 60, Span 80, Span 120; sodium lauryl sulfate; polyethoxylated castor oil; polyethoxylated hydrogenated castor oil, sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N,N-dimethyldodecylamine-N oxide, Hexadecyltrimethyl-ammonium bromide, polyoxyl 10 lauryl ether, Brij, bile salts (sodium deoxycholate, sodium cholate), polyoxyl castor oil, nonylphenol ethoxylate, cyclodextrins, lecithin, methylbenzethonium chloride, carboxylates, sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated, ethoxylated aliphatic alcohol, polyoxyethylene surfactants, carboxylic esters, polyethylene glycol esters, anhydrosorbitol ester and its ethoxylated derivatives, glycol esters of fatty acids, carboxylic amides, monoalkanolamine condensates, polyoxyethylene fatty acid amides, quaternary ammonium salts, amines with amide linkages, polyoxyethylene alkyl and alicyclic amines, N,N,N,N tetrakis substituted ethylenediamines, 2-alkyl 1-hydroxyethyl 2-imidazolines, N-coco 3-aminopropionic acid/sodium salt, N-tallow 3-imino-dipropionate disodium salt, N-carboxymethyl n-dimethyl n-9-octadecenyl ammonium hydroxide and n-cocoamidethyl n-hydroxyethylglycine sodium salt.

Tonicity-adjusting agents, which may be used, comprise, but are not limited to, sodium chloride, potassium chloride, zinc chloride, calcium chloride and mixtures thereof. Other isotonicity-adjusting agents may also include, but are not limited to, mannitol, glycerol, and dextrose and mixtures thereof.

The pH may be adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid. Examples of particularly suitable organic acids are selected from the group consisting of ascorbic acid, citric acid, malic acid, tartaric acid, maleic acid, succinic acid, fumaric acid, acetic acid, formic acid and propionic acid.

Complexing agents according to the present invention may comprise, but are not limited to, editic acid (EDTA) or one of the known salts thereof, e.g. sodium EDTA or disodium EDTA dihydrate (sodium edetate).

An anti-microbial preservative agent may be added for multi-dose packages.

Suitably, the pharmaceutical composition according to the present invention is administered using dosage forms suitable for delivery of drugs to the respiratory tract. However, other dosage forms such as parentral dosage forms may also be envisaged under the ambit of the invention.

The present invention also provides a process to manufacture compositions according to the present invention.

The present invention provides a process of preparing a dry powder inhalation formulation which process comprises admixing of a pharmaceutically acceptable carrier or excipient with the actives and providing the formulation as a dry powder inhaler.

The present invention further provides a method for treatment and/or prophylaxis of influenza which comprises administering a pharmaceutical composition according to the present invention comprising a low dose of zanamivir.

It may be well acknowledged to a person skilled in the art that the said pharmaceutical composition, according to the present invention, may further comprise one or more active in particular antibiotics, anti-bacterial and other anti-viral agents such as those used to treat respiratory infections.

For example, other compounds effective against influenza viruses, such as but not limited to, amantadine, rimantadine, acyclovir, azidothymidine, vidarabine, ribavirin, dapsone, chloramphenicol, neomycin, cefaclor, cefadroxil, cephalexin, erythromycin, clindamycin, lincomycin, amoxicillin, ampicillin, carbenicillin, methicillin, nafcillin, penicillin, polymyxin, tetracycline, amphotericin-b, candicidin, lucensomycin, mepartricin, natamycin, nystatin, griseofulvin, oligomycins, neomycin tubercidin, picloxacillin, penicllins, sulfonamides, cephalosporins, quinolones, or their pharmaceutically acceptable salts, solvates, tautomers, derivatives, enantiomers, isomers, hydrates, prodrugs or polymorphs thereof may be included in such combinations.

The following examples are for the purpose of illustration of the invention only and are not intended in any way to limit the scope of the present invention.

### Example 1

| Sr. No. | Ingredients | Qty/Unit (mg/capsule) |
|---|---|---|
| 1. | Zanamivir | 3.00 |
| 2. | Coarse Lactose | 15.40 |
| 3. | Fine Lactose | 6.50 |
| | Total | 25.00 |

Process:

1) Zanamivir was sifted with fine lactose.

2) The mixture obtained in step (1) was co-sifted with coarse lactose.

3) The mixture obtained in step (2) was blended and filled into capsules.

### Example 2

| Sr. No. | Ingredients | Qty/Unit (mg/capsule) |
|---|---|---|
| 1. | Zanamivir | 4.00 |
| 2. | Coarse Lactose | 14.70 |
| 3. | Fine Lactose | 6.30 |
| | Total | 25.00 |

Process:

1) Zanamivir was sifted with fine lactose.

2) The mixture obtained in step (1) was co-sifted with coarse lactose.

3) The mixture obtained in step (2) was blended and filled into capsules.

To illustrate the Revolizer™ inhalation device, a preferred embodiment thereof will now be described with reference to the accompanying drawings (which in no way restrict the scope of the invention and are for the purpose of illustration only) in which:

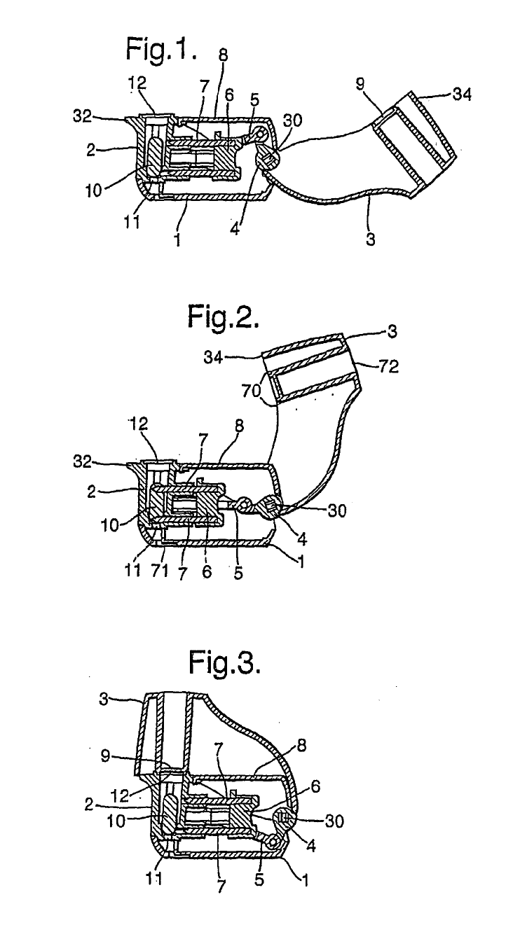

1) FIG. 1 is a cross-sectional view of the inhalation device, with piercing means in a fully retracted position and the inhaler mouthpiece in a fully opened position.

2) FIG. 2 is a cross-sectional view of the inhalation device, with piercing means in a fully extended piercing position and the inhaler mouthpiece in a partially open/partially closed position, and illustrates a preferred position of the vents.

3) FIG. 3 is a cross-sectional view of the inhalation device, with piercing means in a fully retracted position and the inhaler mouthpiece in a fully closed position.

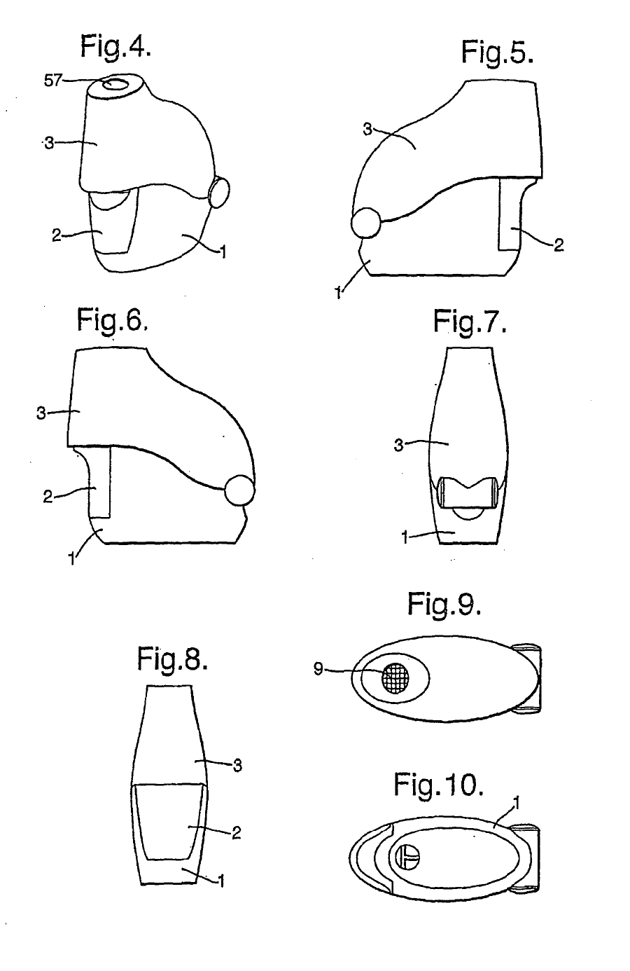

4) FIG. 4 is a perspective view of the exterior of the inhalation device with the mouthpiece in the fully closed position.

5) FIG. 5 is a plan view of a side of the inhalation device with the mouthpiece in the fully closed position.

6) FIG. 6 is a plan view of the other side (with respect to FIG. 5) of the inhalation device with the mouthpiece in the fully closed position.

7) FIG. 7 is a plan view of the rear of the inhalation device with the mouthpiece in the fully closed position.

8) FIG. 8 is a plan view of the front of the inhalation device with the mouthpiece in the fully closed position.

9) FIG. 9 is a top end view of the inhalation device with the mouthpiece in the fully closed position, viewed from the mouthpiece end.

10) FIG. 10 is a bottom end view of the inhalation device with the mouthpiece in the fully closed position, viewed from the lower body end.

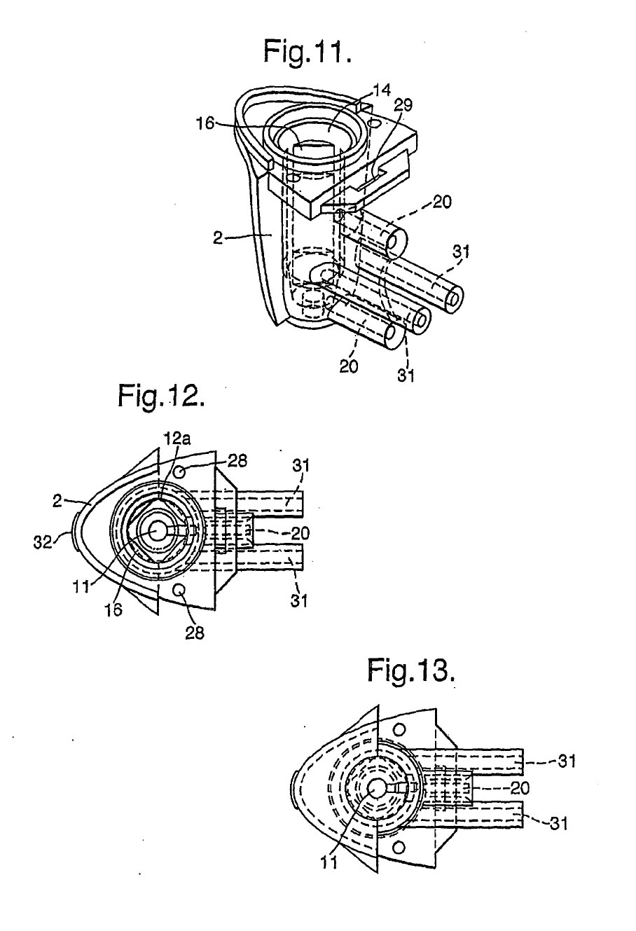

11) FIG. 11 is a perspective view of a holder for a dry powder medicament capsule.

12) FIG. 12 is an end view of the medicament capsule holder, viewed from the air outlet end.

13) FIG. 13 is an end view of the medicament capsule holder viewed from the air inlet end.

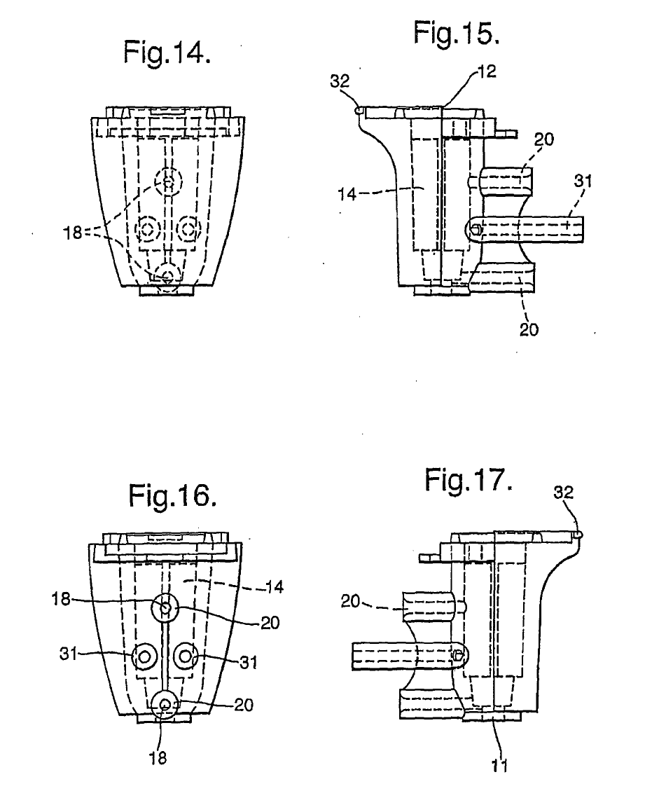

14) FIG. 14 is a front view of the medicament capsule holder.

15) FIG. 15 is a side view of the medicament capsule holder.

16) FIG. 16 is a rear view of the medicament capsule holder.

17) FIG. 17 is a side view of the medicament capsule holder.

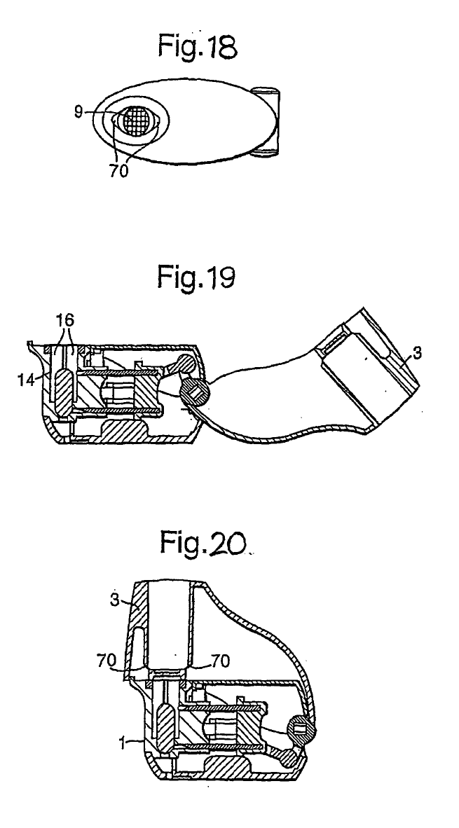

18) FIG. 18 is a top end view of a preferred inhalation device with the mouthpiece in the fully closed position, viewed from the mouthpiece end. It illustrates the position of the vents.

19) FIG. 19 is a cross-section of a preferred inhalation device illustrating projections (16) of increased height compared to FIG. 11.

20) FIG. 20 is a cross-section of a preferred inhalation device and shows a preferred position of the vents.

The Revolizer™ inhalation device comprises a lower body (1) having a medicament or capsule holder (2) to hold the capsule or similar suitable medicament receptacle, and a mouthpiece (3) attached to the lower body (1) by a pivot (30) about which the mouthpiece (3) is rotatable

The medicament or capsule holder (2) comprises a chamber (14) to receive a capsule, an air inlet (11), an air outlet (12), and means for generating turbulence (16). The means for generating turbulence (16) may comprise one or more projection(s), as shown in FIGS. 11 and 12. Preferably, the projections (16) extend to substantially the top of medicament chamber (14) so that they align with the upper rim of the chamber (14), as illustrated in FIG. 19.

The means for generating turbulence (16) have a dual function: they both hold a capsule (10) received within the chamber (14), and generate turbulence in fluid flow through the chamber (14) and around a capsule (10) received therein.

The capsule holder (2) is preferably adapted to receive one or more piercing pins (7) that are moveable between a retracted position in a piercing pin holder (6), shown in FIGS. 1 and 3, and an extended piercing position, shown in FIG. 2. The piercing pins (7) are operated by a link (5) that enables the linear movement of the piercing pin (7) to pierce the capsule or similar suitable medicament receptacle (10) and retract back.

In one embodiment, guide means (20) extend from the openings (18) in a side wall of the chamber (2). The guide means (20) are positioned so as to guide the movement of the piercing pins (7) between fully extended and fully retracted positions. The guide means (20) can be best appreciated from FIGS. 11 to 17.

The mouthpiece (3) pivots laterally to the lower body (1) so as to rotate about an axis and thereby open and close the outlet (12) of the capsule holder (2).

FIG. 2 illustrates the preferred position of the vents (70, the vents themselves are not shown in the drawing). Two crescent-shaped vents are positioned opposing each other on either side of a mesh (9). FIG. 2 also illustrates the primary air inlet (71) on the base of the lower body or housing (1), and the final air outlet (72) on mouthpiece (3). When the device is in the closed position (as in FIG. 3), the device defines an inhalation passage extending through the device between inlet (71) and outlet (72). FIG. 18 illustrates a preferred position of the vents (70) relative to mesh (9). The exact positioning can be appreciated with reference to FIG. 20, which shows the position of vents (70) relative to the mouthpiece (3) and lower body (1).

In use, the mouthpiece (3) is opened, thereby retracting the piercing pins (7), and a capsule or similar suitable medicament receptacle (10) is placed into the chamber (14). The projections (16) loosely hold the capsule (10) within the chamber (14). On closing the mouthpiece (3), the piercing pins (7) pierce the capsule (10) in the capsule holder (2) as shown in FIG. 2. On fully closing the mouthpiece (3), the piercing pins retract from the medicament holder (2).

As air is inhaled by the user from the chamber (14) through the outlet (12) and mouthpiece (3), the projections (16) generate turbulence in the air flow through the chamber (14). Turbulence in the air flowing around the capsule (10) causes vibration of the capsule (10) within the chamber (14), and this vibration enhances the dispersion of medicament contained within the capsule (10). Accordingly, less forceful inhalation by the user is required to liberate a full dose of medicament from the capsule (10).

It will be readily apparent to one skilled in the art that varying substitutions and modifications may be made to the invention disclosed herein. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1. A pharmaceutical composition comprising zanamivir and one or more pharmaceutically acceptable excipients, wherein the total daily dose of the zanamivir is less than 10 mg.

2. A pharmaceutical composition according to claim 1, formulated for administration at least once a day.

3. A pharmaceutical composition according to claim 1, wherein the composition delivers from 3 mg to 8 mg of zanamivir per administered dose.

4. A pharmaceutical composition according to claim 1, formulated for use as a dry powder inhaler (DPI), metered dose inhaler (MDI), nebuliser, nasal spray, nasal drops or insufflation powders.

5. A pharmaceutical composition according to claim 4 formulated for use as a dry powder inhalation formulation.

6. A pharmaceutical composition according to claim 5, further comprising at least one finely divided pharmaceutically acceptable carrier suitable for use in dry powder inhalation formulations.

7. A combination composition according to claim 6, wherein said carrier includes a saccharide and/or a sugar alcohol.

8. A pharmaceutical composition according to claim 5, wherein the pharmaceutical composition is encapsulated in capsules, or is encapsulated in blisters, or is contained within a reservoir in a single-dose or multi-dose dry powder inhalation device.

9. A pharmaceutical composition according to claim 5, wherein the pharmaceutical composition is administered via Revolizer™.

10. A pharmaceutical composition according to claim 1, further comprising one or more active(s) selected from amantadine, rimantadine, acyclovir, azidothymidine, vidarabine, ribavirin, dapsone, chloramphenicol, neomycin, cefaclor, cefadroxil, cephalexin, erythromycin, clindamycin, lincomycin, amoxicillin, ampicillin, carbenicillin, methicillin, nafcillin, penicillin, polymyxin, tetracycline, amphotericin-b, candicidin, lucensomycin, mepartricin, natamycin, nystatin, griseofulvin, oligomycins, neomycin tubercidin, picloxacillin, penicllins, sulfonamides, cephalosporins or quinolones or their pharmaceutically acceptable salts, solvates, tautomers, derivatives, enantiomers, isomers, hydrates, prodrugs or polymorphs thereof.

11. A process for preparing a pharmaceutical composition for administration according to claim 1, which process comprises admixing one or more pharmaceutically acceptable excipients with zanamivir.

12. A method of treating a subject in need thereof, comprising the step of administering to the subject a pharmaceutical composition according to claim 1 for the treatment and/or prophylaxis of viral diseases.

13. A method of treating a subject in need thereof, comprising the step of administering to the subject a pharmaceutical composition according to claim 12 for the treatment and/or prophylaxis of viral diseases caused by one or more of: influenza A and B, parainfluenza, mumps, Newcastle disease, fowl plaque and Sendai virus.

14. A method of treatment and/or prophylaxis of viral diseases comprising administering a therapeutically effective amount of a composition according to claim 1 to a patient in need thereof.

15. A pharmaceutical composition of claim 1, comprising: Zanamivir—3.00 mg/capsule; coarse lactose—15.40 mg/capsule; and fine lactose—6.50 mg/capsule.

16. A pharmaceutical composition of claim 1, comprising: Zanamivir—4.00 mg/capsule, coarse lactose—14.70 mg/capsule, and fine lactose—6.30 mg/capsule.

17. An inhalation device for inhalation of a pharmaceutical composition of claim 1 from a pierceable capsule, the inhaler comprises a housing for receiving a medicament capsule comprised of the pharmaceutical composition of claim 1; closure means for closing the housing, the closure means being moveable relative to the housing; piercing means suitable for piercing the medicament capsule; wherein movement of the closure means relative to the housing causes movement of the piercing means.

18. The inhalation device of claim 17, wherein the housing comprises means to hold a medicament capsule; the holding means preferably comprising a chamber having one or more air inlets and air outlets; at least one air inlet of the chamber constituting a primary air inlet of the device, or the primary air inlet of the device may be provided separately on the device, for example elsewhere on the housing.

19. The pharmaceutical composition of claim 1, wherein the pharmaceutical composition is formulated for administration by a metered dose inhaler.

20. The pharmaceutical composition of claim 19, wherein the metered dose inhaler includes one or more pharmaceutically acceptable excipients selected from: hydrofluoroalkane (HFC/HFA) propellants, co-solvents, bulking agents, non volatile component, buffers/pH adjusting agents, surface active agents, preservatives, complexing agents, or combinations thereof.
